(19) United States
(12) Patent Application Publication
(10) Pub. No.: US 2006/0104857 A1
(43) Pub. Date: May 18, 2006

(54) Sterilization wrap with indicia for placement of medical instrumentation or trays

(76) Inventors: James M. Pigott, Winnetka, IL (US); Francis A. Czajka, Libertyville, IL (US)

Correspondence Address: JENKENS & GILCHRIST, P.C., 225 WEST WASHINGTON, SUITE 2600, CHICAGO, IL, 60606, US

(21) Appl. No.: 11/273,277

(22) Filed: Nov. 14, 2005

Related U.S. Application Data
(60) Provisional application No. 60/628,077, filed on Nov. 15, 2004.

(51) Int. Cl. A61L 2/00

(52) U.S. Cl. 422/1; 422/292

(57) ABSTRACT

A sterilization wrap used to protect articles during and after sterilization is disclosed. The sterilization wrap includes a first sheet bonded to a second sheet to form the sterilization wrap. The sterilization wrap has an inner surface and an outer surface. The inner surface has indicia for assisting a user in placing at least one article to be sterilized in a proper position in contact with the inner surface of the sterilization wrap.

### CROSS-REFERENCE TO RELATED APPLICATIONS

This application claims the benefit of U.S. Provisional Application Ser. No. 60/628,077, filed on Nov. 15, 2004. The disclosure of the aforementioned provisional application is incorporated by reference in its entirety herein.

### FIELD OF THE INVENTION

The present invention relates generally to sterilization wraps used to contain reusable articles during sterilization and to maintain the sterility of the instruments during storage. More particularly, the present invention relates to a sterilization wrap having indicia to aid the user in the proper placement of the reusable medical instruments on the sterilization wrap.

### BACKGROUND OF THE INVENTION

Surgical instruments and other medical equipment are often reusable but must be sterilized before being used in surgical or medical procedures. Smaller pieces of medical equipment, such as surgical instruments, usually are sterilized by subjecting the instruments to steam or ethylene oxide. For years, it has been standard protocol for hospitals and surgical centers to wrap the reusable instruments or a tray containing the instruments in sheet(s) of material, often referred to as a sterilization wrap, before the sterilization process. Steam or ethylene oxide is able to penetrate the sheets of material to sterilize the instruments while bacteria are prevented from entering the sheets of material during storage.

Originally, sterilization wraps were made from muslin cloth. However, one layer of muslin cloth did not provide adequate protection against bacterial infiltration, so two layers were used. The instruments could be wrapped in the two muslin cloths sequentially such that the instruments were wrapped in a first cloth and then wrapped again in a second cloth to create a “package in a package.” Alternatively, the muslin cloths could be wrapped non-sequentially. In non-sequential wrapping, the two cloths were put one on top of each other and the instruments were wrapped in both cloths simultaneously in a one-step process.

Subsequently, cellulose-based sterilization wraps replaced the muslin cloth sterilization wraps. The cellulose-based sterilization wraps were made from cellulose-based, non-woven materials that adequately protected against bacterial infiltration but lacked strength so they often tore as a result of handling before and after the sterilization process. As a result, two layers of these early cellulose-based, non-woven materials were used.

Today, sterilization wraps often are made from non-woven materials comprising thermally-bonded layers of spunbond/meltblown/spunbond, commonly referred to as SMS. The spunbond layers provide strength while the meltblown layer prevents bacteria infiltration after sterilization. Sterilization wraps made from non-woven materials are generally disposable.

The process of sequentially double-wrapping instruments is time-consuming and cumbersome because nurses or other hospital personnel must wrap the instruments in one sheet and then place that package on a second sheet and wrap again. Even if the non-sequential wrap method is used, the two individual sheets of the sterilization wrap must be properly aligned before and during wrapping to provide the best environment for sterilizing and storing the sterilized instruments. In order to avoid these processes, sterilization wraps have been developed having multiple sheets that are bonded together to form the sterilization wrap. The individual sheets of these multi-sheet sterilization wraps can be bonded to one another in a variety of bond configurations to provide two or more layers of protection. For example, the sheets can be bonded together along the edges of the sheets and substantially not bonded in the central portion of the sheets. The use of a sterilization wrap comprising two sheets bonded together reduces the time it takes to wrap and unwrap instruments and makes the overall wrapping process easier.

Sterilization wraps are produced and stocked in a number of sizes to accommodate the different sizes and shapes of medical instruments. To assist the user in distinguishing the size of the sterilization wrap, different sized wraps are often different colors. Although a color-coding system is helpful, it may still be difficult to determine whether a particular wrap is the correct size for an instrument or instrument tray. If the sterilization wrap is too small, there is a greater risk that the wrap will tear during the wrapping process, thereby, requiring the wrap to be discarded and a second wrap used. Furthermore, if the wrap is too small, there is a greater risk that the wrap will tear during handling either before or after the sterilization process which may ultimately lead to the sterility of the instruments being compromised. On the other hand, the use of wraps that are too large for an instrument or tray to be sterilized may increase wrapping and unwrapping time as a result of the extra material. In addition, the overall cost of the sterilization process increases because larger sterilization wraps cost more than smaller sterilization wraps.

Although the prior art sterilization wraps may be adequate for their intended purpose, there exists a need for improved sterilization wraps. Specifically, there exists a need for improved sterilization wraps that further reduce the amount of time it takes to wrap the instruments and allow the user to quickly determine whether they have selected the correct size wrap for the article(s) to be sterilized.

### SUMMARY OF THE INVENTION

According to one embodiment of the present invention, a sterilization wrap used to protect articles during and after sterilization is disclosed. The sterilization wrap includes a first sheet bonded to a second sheet to form the sterilization wrap. The sterilization wrap has an inner surface and an outer surface. The inner surface has indicia for assisting a user in placing at least one article to be sterilized in a proper position in contact with the inner surface of the sterilization wrap. The article or articles may be medical instrument(s) or medical instrument tray(s) containing one or more medical instruments.

According to another embodiment of the present invention, a method of wrapping an article for sterilization is disclosed. The method comprises the step of providing a sterilization wrap comprising at least one sheet. The sheet includes indicia for determining the proper placement of at least one article to be sterilized. The method further comprises the steps of placing the at least one article to be sterilized within the indicia, wrapping the at least one article in the sterilization wrap, and securing the sterilization wrap around the at least one article. The article or articles may be medical instrument(s) or medical instrument tray(s) containing one or more medical instruments.

In yet another embodiment of the present invention, a method for determining the correct size of a sterilization wrap for an article to be sterilized is disclosed. In particular, a sterilization wrap having indicia corresponding to the maximum size of the article that can be sterilized in the sterilization wrap is disclosed. An article to be sterilized is then placed in the sterilization wrap. If the article extends beyond the indicia, the article is removed from the sterilization wrap and the next larger size sterilization wrap is used. However, if the article is the same size or only slightly smaller than the indicia on the sterilization wrap, the article is wrapped for sterilization. The article or articles may be medical instrument(s) or medical instrument tray(s) containing one or more medical instruments.

The above summary of the present invention is not intended to represent each embodiment, or every aspect, of the present invention. Additional features and benefits of the present invention are apparent from the detailed description, figures, and claims set forth below.

### BRIEF DESCRIPTION OF THE DRAWINGS

FIG. 1 is a top plan view of one embodiment of the sterilization wrap of the present invention.

FIG. 2 is an exploded perspective view of the sterilization wrap of FIG. 1, according to one embodiment of the present invention.

FIG. 3 is a cross-sectional view of the sterilization wrap shown in FIG. 1 taken along line 3-3.

FIG. 4 is a perspective view of the embodiment of the sterilization wrap shown in FIG. 1 with a sterilization tray.

FIG. 5 is a top plan view of a sterilization wrap with a sterilization tray, according to another embodiment of the present invention.

FIG. 6a is an upper perspective view of the sterilization wrap of the FIG. 1 prior to being folded around a sterilization tray, according to one embodiment of the present invention.

FIG. 6b is an upper perspective view of a first corner portion of the sterilization wrap of FIG. 6a being folded around a sterilization tray, according to one embodiment of the present invention.

FIG. 6c is an upper perspective view of a tip of the first corner portion of FIG. 6b being folded back, according to one embodiment of the present invention.

FIG. 7 is a top plan view of a sterilization wrap, according to yet another embodiment of the present invention.

While the invention is susceptible to various modifications and alternative forms, specific embodiments have been shown by way of example in the drawings and will be described in detail herein. It should be understood, however, that the invention is not intended to be limited to the particular forms disclosed. Rather, the invention is to cover all modifications, equivalents, and alternatives falling within the spirit and scope of the invention as defined by the appended claims.

### DESCRIPTION OF THE ILLUSTRATED EMBODIMENTS

While the present invention is described with respect to its use with reusable medical instruments, those skilled in the art will understand that the sterilization wrap of the present invention may be used in connection with any article that requires sterilization before use.

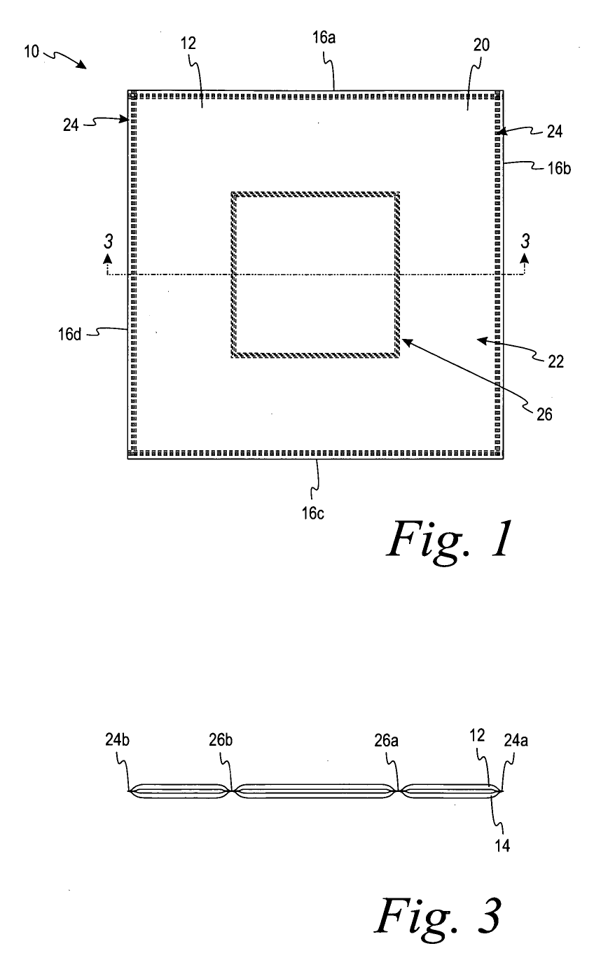

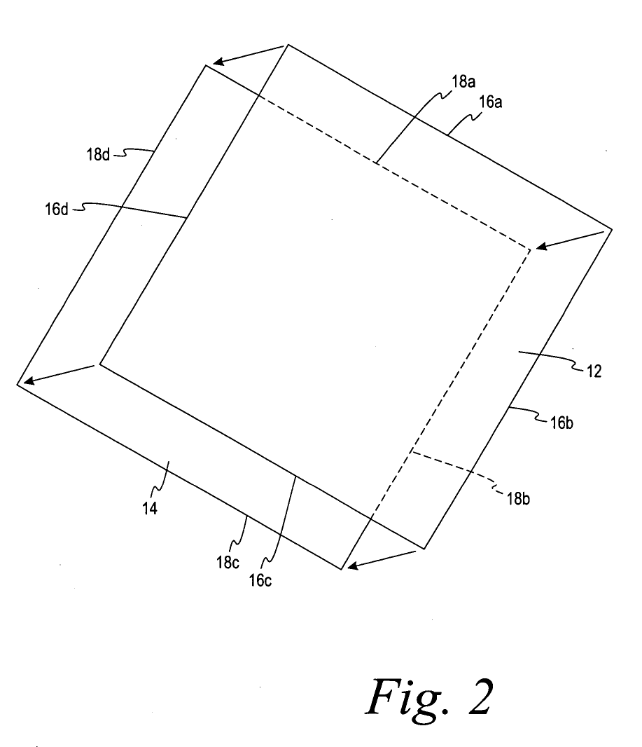

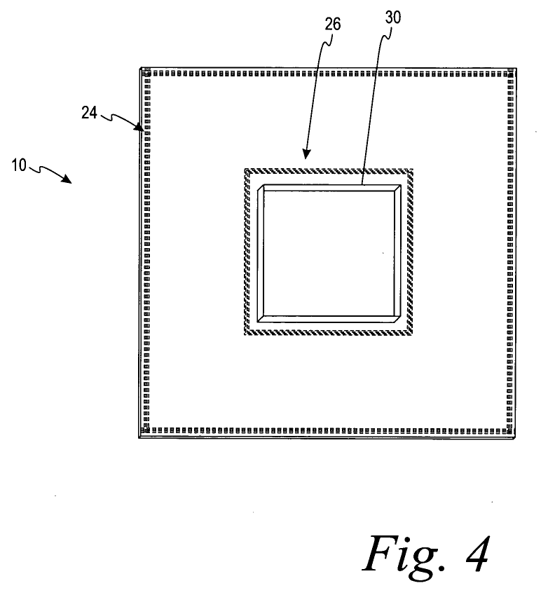

Referring now to FIGS. 1-3, according to one embodiment of the present invention, a sterilization wrap 10 is illustrated. The sterilization wrap 10 is formed from two sheets of material that are bonded together. In particular, a first sheet 12 and a second sheet 14 are placed, one on top of the other, so that the edges 16a-d of the first sheet 12 are substantially aligned with the edges 18a-d of the second sheet 14, as best illustrated in FIG. 2. Once aligned, the first sheet 12 and the second sheet 14 are attached, forming bond 24, along substantially the entire length of their edges 16,18. The first sheet 12 forms an inner surface 20 of the sterilization wrap 10, and the second sheet 14 forms an outer surface (not shown). During use, the inner surface 20 of the sterilization wrap 10 contacts the article or articles to be sterilized. In alternative embodiments, the sterilization wrap is a single sheet of material that forms both the inner surface and the outer surface.

As illustrated, the bond 24 is located generally near the edges 16,18 of the sheets 12,14. In some embodiments, the bond 24 may be located along only two parallel edges (e.g., 16a,18a and 16c,18c only) of the sheets 12,14. In further embodiments, the bond 24 may be located along only one edge of the sheets 12,14. In other embodiments, the bond 24 may be located along only three edges while in still other embodiments, the bond 24 may be located along all four edges of the sheets 12,14. The bond 24 may extend along the entire length or substantially the entire length of the edges 16,18 or may extend along less than or substantially less than the entire length of the edges 16,18.

The bond 24 may be formed in a variety of ways so as to attach the first sheet 12 to the second sheet 14. The sheets 12,14 may be attached together by any method known in the art that will withstand the sterilization process. Accordingly, the sheets 12,14 may be bonded by, for example, stitching, such as a surged seam, adhesive bonding, ultrasonic bonding, thermal bonding, such as with a bar sealer, or any other suitable attachment method(s). In addition, the bond 24 may be one continuous bond or a plurality of distinct bonds. The bond 24 can be located in any suitable location to attach the first sheet 12 to the second sheet 14 but is generally located at the edges 16,18 or generally adjacent the edges 16,18.

The inner surface 20 and the outer surface of the sterilization wrap 10 may be visually identical, that is, the inner and outer surfaces cannot be differentiated on visual inspection. Alternatively, the inner surface 20 and the outer surface of the sterilization wrap 10 may be visually distinct. For example, the inner surface 20 and the outer surface may be different colors, may be imprinted with different patterns or words, or may have visually distinct textures.

As further shown in FIGS. 1-3, a center portion 22 of the sterilization wrap 10 contains indicia or markers for assisting the user in placing the instrument(s) or the instrument tray(s) in or near the center of the sterilization wrap 10. In the illustrated embodiment, a set of guide lines 26 are provided to assist in placing one or more instruments or instrument trays in contact with the sterilization wrap 10. The indicia may be selected from a variety of shapes including, but not limited to, a circle, a square, a triangle, a rectangle, and a trapezoid. The indicia may, however, be any shape, regular or irregular, sufficient to indicate to a user where the instrument(s) or instrument tray(s) should be positioned.

In embodiments where indicia or markers are utilized such as, but not limited to, guide lines, the indicia may serve several important functions. First, the indicia assist the user in placing the instrument(s) or instrument tray(s) in or near the center of the sterilization wrap 10 prior to wrapping. If the instrument(s) or tray(s) is not centered on the sterilization wrap 10 prior to folding, gaps or openings may occur in the wrapped package which may lead to contamination of the sterilized instruments. Further, by assisting the user in accurately placing the instrument(s) or tray(s) the first time, the user is able to place and wrap the instrument(s) or tray(s) more quickly. In addition, the indicia allow the user to quickly and easily determine if a sterilization wrap of the correct size is being used. For example, if the instrument(s) or instrument tray(s) to be sterilized extends beyond the indicia, a larger sterilization wrap is needed. Conversely, if the instrument(s) or tray fits well within the indicia, a smaller sterilization wrap can be used. By assisting the user in initially selecting a sterilization wrap of the correct size, the indicia help save money as the user will not unnecessarily select too large of wraps which cost more money or too small of wraps which may tear during the wrapping process thereby resulting in the discarding of the wrap and the unnecessary use of a second wrap.

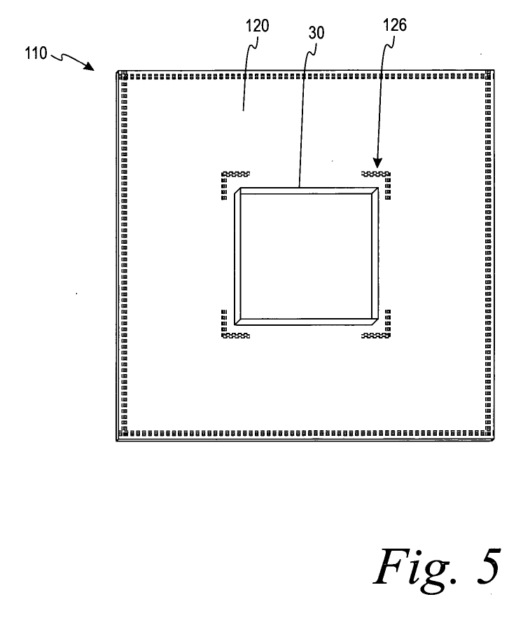

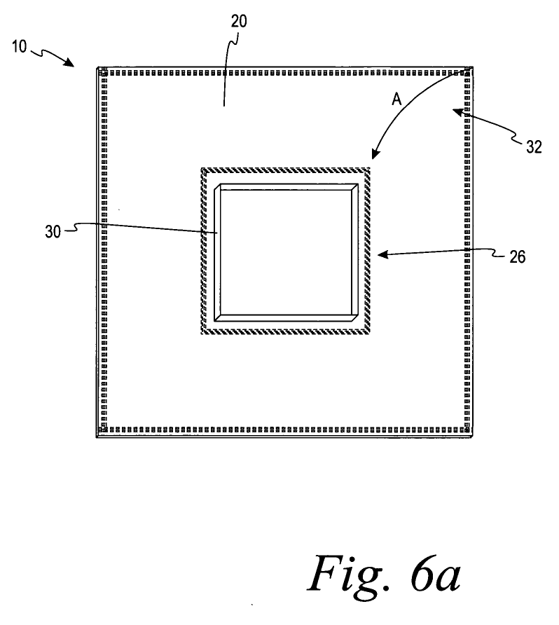

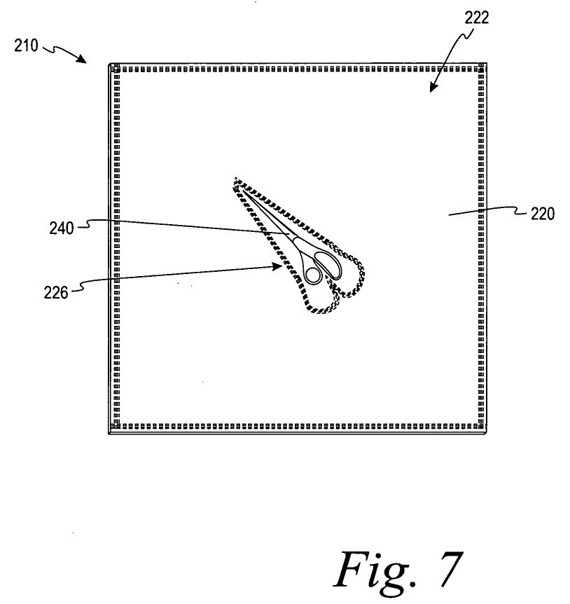

As shown in FIGS. 1 and 4, the guide lines 26 may form an outline of, for example, a sterilization tray 30 (FIG. 4). Alternatively, the guide lines 26 may form an outline of a specific instrument or group of instruments, as will be more fully described with respect to FIG. 7. The guide lines 26 may be placed on the first and second sheets 12,14 or only on the inner surface 20 of the first sheet 12 of the sterilization wrap 10. The guide lines 26 may be created on the first and/or second sheets 12,14 by any method known in the art, including but not limited to, printing or imprinting the lines of the guide lines 26 on the first and/or second sheets 12,14. In one embodiment, the guide lines 26 are printed on the sheets using a colored adhesive ink. As shown in FIGS. 1-2, in one embodiment, the guide lines 26 are formed by ultrasonic bonds used to assist in bonding the first sheet 12 to the second sheet 14 to form the sterilization wrap 10. Although the guide lines in FIGS. 1 and 4 are shown as a complete outline of a sterilization tray, a set of guide lines 126 located on a sterilization wrap 110 may show only a portion (e.g., two or more corners or corner portions) of the outline of a sterilization tray 30 such as in FIG. 5.

In some embodiments of the present invention, the sterilization wraps comprise two or more sheets of material bonded together. In other embodiments, the sterilization wrap comprises a single sheet of material. In yet other embodiments, the sterilization wrap comprises any plurality of single sheets of material which are not bonded together. Where a plurality of sheets of material are used, the sheets are generally the same size and are positioned one on top of the other so that the edges of the sheets are substantially aligned. The substantially aligned plurality of sheets of material may be bonded together using any suitable attachment method or mechanism as further described above.

Typically, the sheets are square or rectangular in shape, though it is apparent to those skilled in the art that other sheet shapes may be utilized. The sheets may be produced in a variety of sizes to accommodate medical instruments and trays having different sizes and shapes. For example, the sheets may be about 24 in.×24 in. (61 cm×61 cm), about 48 in.×48 in. (122 cm×122 cm), about 50 in.×72 in. (127 cm×183 cm), etc. As is apparent to those skilled in the art, any size sheet(s) may be used in accordance with the present invention. Where two or more sheets are bonded together, any bonding pattern may be used as long as a sufficient amount of steam or ethylene oxide is able to penetrate the bonded sheets and sterilize the instruments.

Each sheet of the sterilization wrap 10 may be made from a variety of materials including woven fabrics, non-woven fabrics, tissue, paper, and polyethylene plies. Generally, disposable wraps are preferred for sterilization of articles. Disposable wraps are typically created from a plurality of non-woven fibers. The fibers can be natural fibers or may be synthetically produced. For example, fibers can be used that are made from polyolefin, polyester, rayon, cellulose, or the like. A variety of processes may be used to create non-woven fabrics from the plurality of fibers for use in the sterilization wraps of the present invention. For example, the non-woven fabrics may be made by spunbonding, meltblowing, hydroentangling, wet laying, air laying, solution spinning, or the like.

In one embodiment, the material used to make an individual sheet of the sterilization wrap 10 is a non-woven SMS. The two outer spunbond layers may be made from extruded polyolefin fibers laid in a random pattern and then bonded together. The meltblown layer may also be made from extruded polyolefin fibers, though the fibers in the meltblown layer are generally smaller that the fibers in the spunbond layer. The outer spunbond layers and the inner meltblown layer are then laminated together to from the SMS material.

In another embodiment, the material used to make an individual sheet of the sterilization wrap 10 is a nonwoven four-layer laminate comprising first and second spunbond layers and third and fourth meltblown layers positioned between the spunbond layers with all of the layers being bonded together. This material is commonly referred to as SMMS. As discussed, the fibers forming the spunbond and meltblown layers of both the SMS and the SMMS are generally polyolefin fibers and include, but are not limited to, polyethylene and polypropylene. Those skilled in the art will understand that SMS and SMMS may be made from a variety of polyolefins and combinations thereof that are suitable for use in a steam and/or ethylene oxide sterilization process. Both SMS and SMMS are commercially available from a number of sources including CNC International, Ltd. in Thailand.

In some embodiments of the present invention, the sterilization wrap 10 includes two individual sheets being bonded together. In these embodiments, the first sheet and the second sheet may be SMS, SMMS, or any other suitable material. The first and second sheet may be made of the same material or may be made from different materials. In other embodiments of the sterilization wrap, a third sheet may be positioned between the first sheet and the second sheet. The third sheet may comprise SMS, SMMS, spunbond polyethylene, or any other suitable material. In yet other embodiments, the sterilization wrap includes additional sheets and the additional sheets may be made of the same material or may comprise any number of suitable materials, as is known in the art.

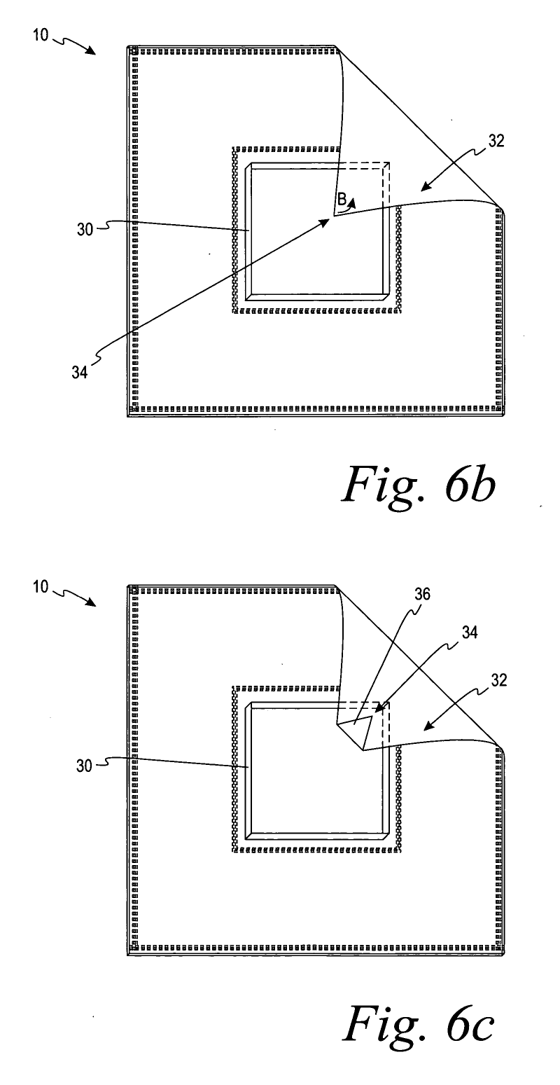

In use, as illustrated in FIGS. 4-5, respectively, a sterilization tray 30 is placed on the inner surface 20, 120 of the sterilization wrap 10, 110 within the guide lines 26, 126. The sterilization wrap 10, 110 is then folded around the sterilization tray 30 using the standard “envelope” method, as is well known in the art and is illustrated in FIGS. 6a-6c. As illustrated in FIG. 6a, the sterilization tray 30 is placed in contact with the inner surface 20 of the sterilization wrap 10 within the guide lines 26. A first corner portion 32 of the sterilization wrap 10 is folded in the direction of arrow A. The first corner portion 32 is folded over a portion of the sterilization tray 30, as shown in FIG. 6b. Subsequently, a tip 34 of the first corner portion 32 is folded back in the direction of arrow B to form a point 36, as illustrated in FIG. 6c. Additional indicia may placed on the sterilization wrap 10 to indicate the location at which the tip 34 of the first corner portion 32 of the sterilization wrap 10 should be folded back to form the point 36. This same technique is then used to fold over the remaining corner portions of the sterilization wrap 10.

Once all four of the corner portions of the sterilization wrap 10 have been folded as described above, a package is created around the instrument(s) and/or instrument tray(s) by the sterilization wrap 10. The package is then secured by any suitable securing means or methods. For example, the package may be secured with a piece of adhesive tape. At this point, the package may be sterilized as is known in the art such as by use of steam or ethylene oxide.

Turning now to FIG. 7, a sterilization wrap 210 is illustrated according to another embodiment of the present invention. The sterilization wrap 210 includes indicia located in or near a center portion 222 of an inner surface 220 of the sterilization wrap 210. The indicia in the illustrated embodiment form a guide line 226 in the shape of a medical instrument. The guide line 226 may be, for example, representative of a pair of surgical scissors 240, in the illustrated embodiment, or may be representative of any other individual piece—or multiple pieces—of medical instrumentation.

While the present invention has been described with reference to one or more particular embodiments, those skilled in the art will recognize that many changes may be made thereto without departing from the spirit and scope of the present invention. Each of these embodiments and obvious variations thereof is contemplated as falling within the spirit and scope of the invention, which is set forth in the following claims.

What is claimed is:

1. A sterilization wrap comprising:
a first sheet bonded to a second sheet to form the sterilization wrap, the sterilization wrap having an inner surface and an outer surface, the inner surface having indicia for assisting a user in placing at least one article to be sterilized in a proper position in contact with the inner surface.

2. The sterilization wrap of claim 1, wherein the indicia corresponds to the shape of an instrument sterilization tray.

3. The sterilization wrap of claim 1, wherein the indicia corresponds to the shape of at least one medical instrument to be sterilized.

4. The sterilization wrap of claim 1, wherein the indicia corresponds to the shape of the at least one article to be sterilized.

5. The sterilization wrap of claim 1, wherein the first sheet comprises a nonwoven material of polyolefin fibers.

6. The sterilization wrap of claim 5, wherein the second sheet comprises a nonwoven material of polyolefin fibers.

7. The sterilization wrap of claim 1, wherein the first sheet comprises spunbond/meltblown/spunbond.

8. The sterilization wrap of claim 7, wherein the second sheet comprises spunbond/meltblown/spunbond.

9. The sterilization wrap of claim 1, wherein the first sheet comprises spunbond/meltbown/meltblown/spunbond.

10. The sterilization wrap of claim 9, wherein the second sheet comprises spunbond/meltbown/meltblown/spunbond.

11. The sterilization wrap of claim 1 further comprising, a third sheet positioned between the first sheet and the second sheet, the third sheet being bonded to the first and second sheets.

12. The sterilization wrap of claim 1, wherein the outer surface has indicia.

13. The sterilization wrap of claim 1, wherein the first and second sheets are imprinted with different patterns.

14. The sterilization wrap of claim 1, wherein the first and the second sheets are visually identical.

15. The sterilization wrap of claim 1, wherein the first and second sheets are visually distinguishable.

16. The sterilization wrap of claim 1, wherein the first and second sheets are different colors.

17. The sterilization wrap of claim 1, wherein the indicia is one or more of a circle, a square, a triangle, a rectangle, and a trapezoid.

18. A method of wrapping an article for sterilization comprising the steps of:
providing a sterilization wrap, the sterilization wrap comprising at least one sheet, the sheet including indicia for determining the proper placement of at least one article to be sterilized;
placing the at least one article to be sterilized within the indicia;
wrapping the at least one article in the sterilization wrap; and
securing the sterilization wrap around the at least one article.

19. The method of claim 18, wherein the at least one article to be sterilized is one or more of
(i) a reusable medical instrument;
(ii) a plurality of reusable medical instruments; and
(iii) a sterilization tray adapted to contain at least one reusable medical instrument.

20. The method of claim 18, wherein the at least one sheet is bonded to a second sheet of material.

21. The method of claim 20, wherein the at least one sheet and the second sheet have the same composition.

22. The method of claim 20, wherein the at least one sheet and the second sheet comprise spunbond/meltblown/spunbond.

23. The method of claim 20, wherein the at least one sheet and the second sheet comprise spunbond/meltblown/meltblown/spunbond.

24. The method of claim 20, wherein the at least one sheet and the second sheet each have a different composition.

25. The method of claim 20, wherein a third sheet is positioned between and bonded to the at least one sheet and the second sheet.

26. The method of claim 20, wherein the third sheet comprises spunbond polyethylene.
